(19) United States
(12) Patent Application Publication
(10) Pub. No.: US 2016/0256449 A1
(43) Pub. Date: Sep. 8, 2016

(54) H3 RECEPTOR ANTAGONIST FOR USE IN THE TREATMENT OF ALZHEIMER'S DISEASE

(71) Applicant: SANOFI, Paris (FR)

(72) Inventors: Sophie CLAUDEL, Montrouge (FR); Jeanne STEMMELIN, Montrouge (FR); Pascal BARNEOUD, Paris (FR); Philippe DELAY-GOYET, Paris (FR); Mathilde LOPEZ-GRANCHA, Massy (FR); Jeremy PRATT, Paris (FR)

(21) Appl. No.: 15/063,977

(22) Filed: Mar. 8, 2016

Related U.S. Application Data
(63) Continuation of application No. PCT/EP2014/069168, filed on Sep. 9, 2014.

(30) Foreign Application Priority Data
Sep. 9, 2013 (EP) 13306228.1

(51) Int. Cl. A61K 31/4725; A61K 31/445

(52) U.S. Cl. CPC A61K 31/4725; A61K 31/445

(57) ABSTRACT

The disclosure relates to the compound 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt thereof, intended for the treatment of Alzheimer's disease and other types of dementia.

This application is a continuation of International Patent Application No. PCT/EP2014/069168, filed Sep. 9. 2014, which is incorporated herein by reference; and claims priority to European Application No. 13306228.1, filed Sep. 9, 2013.

The present invention relates to the compound 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt thereof for use in the treatment of Alzheimer's disease.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure.

Progressive decline in cognitive performance and disabilities in the activities of daily living (ADL) are key characteristics of Alzheimer's disease, and improving cognitive function in AD (including memory, attention or executive function), functional abilities and behavior represents a complex challenge, given the involvement of numerous neurotransmitters systems and brain regions controlling these functions.

There are several treatments for symptoms of AD currently available. The drugs currently used for treating AD, including memantine and the cholinesterase inhibitors, provide only a minimal benefit to a subset of patients and for a limited period. Thus, there is a large unmet need for better and safer treatments for AD. A new therapeutic strategy eagerly sought for AD patients is to target an improvement as adjunct to existing therapies that would bring additional relief for patients and caregivers.

The histamine H3 receptors are found in the central and peripheral nervous systems. The administration of histamine H3 receptor ligands may influence the secretion of neurotransmitters (e.g., histamine, acetylcholine, monoamines, glutamate, GABA) in the brain and the periphery, and those have been considered appropriate candidates for symptomatic treatment of cognitive disorders, including AD and other dementias.

The compound 2-(cyclohexylmethyl)-N-{2-[(2S)-1-methylpyrrolidin-2-yl]ethyl}-1,2,3,4-tetrahydroisoquinoline-7-sulfonamide is a histamine H3 receptor antagonist with a high affinity and selectivity for the human H3 receptor.

A cooperation between the pharmacological cholinergic and histaminergic-releasing properties of 2-(cyclohexylmethyl)-N-{2-[(2S)-1-methylpyrrolidin-2-yl]ethyl}-1,2,3,4-tetrahydroisoquinoline-7-sulfonamide in the prefrontal cortex and the main property of cholinesterase inhibitors to promote cholinergic tone could support an increased benefit in cognition, functional capabilities and behavior.

The present invention provides clinical evidence that 2-(cyclohexylmethyl)-N-{2-[(2S)-1-methylpyrrolidin-2-yl]ethyl}-1,2,3,4-tetrahydroisoquinoline-7-sulfonamide has positive, yet unpredictable, effects on various aspects of cognition, functional capabilities and behavior in patients on stable treatment with a cholinesterase inhibitor.

### SUMMARY OF THE INVENTION

The present invention relates to combinations of 2-(cyclohexylmethyl)-N-{2-[(2S)-1-methylpyrrolidin-2-yl]ethyl}-1,2,3,4-tetrahydroisoquinoline-7-sulfonamide, or a pharmaceutically acceptable salt thereof, and a cholinesterase inhibitor.

Another aspect of the invention is the compound 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt for use in the adjunct treatment of Alzheimer's disease in a patient on stable treatment with a cholinesterase inhibitor.

Another aspect of the invention is the compound 2-(cyclohexylmethyl)-N-{2-[(2S)-1-methylpyrrolidin-2-yl]ethyl}-1,2,3,4-tetrahydroisoquinoline-7-sulfonamide for use in combination with or as an adjunct to a cholinesterase inhibitor for the treatment of Alzheimer's disease in a patient.

Another aspect of the invention are methods of treating Alzheimer's disease comprising administering to a patient in need thereof a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide in combination with or as an adjunct to a cholinesterase inhibitor.

Another aspect of the invention is the use of a combination of 2-(cyclohexylmethyl)-N-{2-[(2S)-1-methylpyrrolidin-2-yl]ethyl}-1,2,3,4-tetrahydroisoquinoline-7-sulfonamide and a cholinesterase inhibitor for the treatment of Alzheimer's disease.

In yet another aspect, the invention relates to the use of the combinations or adjunct therapies as described herein in the preparation of a medicament for the treatment of Alzheimer's disease.

### BRIEF DESCRIPTION OF THE DRAWINGS

FIG. 1 illustrates the results of the effect of a co-treatment with 2-(cyclohexyl methyl)-N-{2-[(2S)-1-methyl pyrrolidin-2-yl]ethyl}-1,2,3,4-tetrahydroisoquinoline-7-sulfonamide and donepezil on recognition memory in normal mice at various delays using the object recognition task.

FIG. 2 illustrates the results of the effect of a co-treatment with 2-(cyclohexylmethyl)-N-{2-[(2S)-1-methylpyrrolidin-2-yl]ethyl}-1,2,3,4-tetrahydroisoquinoline-7-sulfonamide and donepezil on recognition memory deficit in scopolamine-treated mice using the object recognition task.

FIG. 3 illustrates the change from baseline at week 4, 12, and 24 weeks and 10 weeks after treatment discontinuation (FUP) in the ADAS-Cog 11 items for the four dose groups during the clinical study described in Example 2, in the modified intent-to-treat (mITT) population.

FIG. 4 illustrates the change from baseline at week 12 and week 24 and 10 weeks after treatment discontinuation in the ADCS-ADL for the four dose groups during the clinical study described in Example 2, in the mITT population.

FIG. 5 illustrates the change from baseline at week 8 and week 24 in CDR-S power of attention for the four dose groups during the clinical study described in Example 2, in the mITT population.

FIG. 6 illustrates the change from baseline at week 12 and week 24 and 10 weeks after treatment discontinuation in AES-I apathy score for the four dose groups during the clinical study described in Example 2, in the mITT population.

FIG. 7 illustrates the rate of “responders” at week 24 for the four dose group according to a combined criterion on the ADAS-Cog (2 points improvement vs baseline), ADCS-ADL (no worsening) and CGIC (no worsening) during the clinical study described in Example 2, in the mITT population.

FIG. 8 illustrates the time course for the change from baseline in episodic memory for all four dose groups through week 24 during the clinical study described in Example 2, in the mITT population.

### DETAILED DESCRIPTION OF THE INVENTION

As used above, and throughout the description of the invention, the following terms, unless otherwise indicated, shall be understood to have the following meanings:

“Active Ingredient” as used herein, may be 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt thereof, a hydrate or solvate thereof, or a solvate or hydrate of a pharmaceutically acceptable salt; or a cholinesterase inhibitor, such as donepezil or a pharmaceutically acceptable salt thereof.

The abbreviation “API” as used herein, refers to 2-(cyclohexylmethyl)-N-{2-[(2S)-1-methylpyrrolidin-2-yl]ethyl}-1,2,3,4-tetrahydroisoquinoline-7-sulfonamide difumarate monohydrate.

“ADAS-Cog” as used herein, refers to a sensitive and standardized neuropsychological test battery to assess cognitive dysfunction characteristics of patients with AD including memory and orientation, language (aphasia), motor skills (praxis), attention and concentration. The original, standard version consists of 11 items. The total score of these 11-item scores ranges from 0-70 with higher scores indicating greater cognitive impairment. More recently, two additional tasks have been added in a new 13-item version of the scale proposed for patients with mild disease: Delayed Word Recall and Concentration/Distractibility.

“ADCS-ADL” as used herein, refers to the Alzheimer's Disease Cooperative Study Activities of Daily Living Inventory, which is a comprehensive battery of ADL questions used to measure the functional capabilities of patients. The total score ranges from 0 (worst possible) to 78 (best possible).

“AES-I” as used herein, refers to the Apathy Evaluation Scale for Informant which assess global measure of apathy. Scores range from 18 (best possible) to 72 (worst possible).

“CDR-S” as used herein, refers to the Cognitive Drug Research computerized assessment system which tests various aspects of human cognitive function, memory, and attention.

“CGIC” as used herein, refers to the Clinical Global impression of Change assessed (at week 12 and week 24 during the clinical study described in Example 2) by the investigator according to 3 modalities: “Improvement” (includes marked, moderate and minimal improvement), “No change” and “Worsening” (includes minimal, moderate and marked worsening).

Actigraphy as used herein refers to an assessment of activity measured by an Actiwatch®, which was worn day and night by the patient during 4 periods of 15 days: before randomization, immediately after randomization, around week 12 and after treatment discontinuation around week 34 during the clinical study described in Example 2. The watch measures movements translating into assessment of sleep quality (number and duration of awakenings, wake after sleep onset or WASO, fragmentation index, sleep efficiency, total sleep period, total sleep time) and circadian rhythms (interdaily stability and intradaily variability, relative amplitude).

“Adjunctive therapy,” and “Adjunct therapy” as used herein, means the treatment or administration of 2-(cyclohexylmethyl)-N-{2-[(2S)-1-methylpyrrolidin-2-yl]ethyl}-1,2,3,4-tetrahydroisoquinoline-7-sulfonamide, or a pharmaceutically acceptable salt thereof, together with a primary treatment. In one aspect of the invention, the primary treatment comprises treatment with a cholinesterase inhibitor. In one aspect, the primary treatment comprises treatment with a compound selected from the group consisting of galantamine, rivastigmine, donepezil, mimopezil, ladostigil, and huperzine A. In one aspect, the primary treatment comprises treatment with a with a cholinesterase inhibitor selected from the group consisting of donepezil (brand name ARICEPT®), galantamine (RAZADYNE®), and rivastigmine (branded as EXELON® and EXELON® PATCH). In one aspect, the primary treatment comprises treatment with the cholinesterase inhibitor donepezil.

“Donepezil” is a reversible inhibitor of acetylcholinesterase, known chemically as (±)-2,3-dihydro-5,6-dimethoxy-2-[[1-(phenylmethyl)-4-piperidinyl]methyl]-1H-inden-1-one, and is sold in the United States as a hydrochloride salt for example under the brand name ARICEPT®.

The abbreviation “DNP” as used herein refers to donepezil hydrochloride.

“Patient” or “subject” includes any mammal. Preferably, the mammal is human.

“Patient in need of treatment” as used herein includes patients with AD or other type of dementia including a patient 1) who is already diagnosed, at any clinical stage, including a patient having mild cognitive impairment to advanced dementia; and/or 2) who has early or prodromal symptoms and signs of AD and/or 3) who has been diagnosed as susceptible to AD due to age, hereditary, a measurable biomarker, or other factors for whom a course of treatment is medically recommended to delay the onset or evolution or aggravation or deterioration of the symptoms or signs of disease.

As used herein, a patient with “preclinical Alzheimer's disease” is one for whom the diagnosis is based on a measurable biomarker of Alzheimer's disease or the detection of subtle evidence of cognitive deficits, without functional impairment.

As used herein, a patient with “mild cognitive impairment” is one for which a cognitive deficit is present, but without sufficient impairment to constitute dementia.

As used herein, a patient with “mild to moderate Alzheimer's disease” is one for whom the diagnosis is based on Dementia of Alzheimer Type DSM-IV criteria and NINCDS/ADRDA criteria for Probable AD (National Institute of Neurological and Communicative Disorders and Stroke/AD and Related Disorders Association) with a MiniMentalState Examination (MMSE) score ≧10 and ≦25 and a Clinical Dementia Rating global score=0.5, 1, or 2. Within this population, a patient with “mild Alzheimer's disease” has an MMSE score ≧20, and a patient with “moderate Alzheimer's disease” has an MMSE score of <20.

As used herein, a patient with “moderate to severe Alzheimer's disease” is one for whom the diagnosis is based on Dementia of Alzheimer Type DSM-IV criteria and NINCDS/ADRDA criteria for Probable AD (National Institute of Neurological and Communicative Disorders and Stroke/AD and Related Disorders Association) with a MiniMentalState Examination (MMSE) score below 19 and a Clinical Dementia Rating global score=2 or 3.

As used herein, a patient with “dementia of Alzheimer Type” (DAT) refers to a patient suffering from Alzheimer's disease. Diagnosis is based on Dementia of Alzheimer Type DSM-IV criteria and NINCDS/ADRDA criteria for Probable AD (National Institute of Neurological and Communicative Disorders and Stroke/AD and Related Disorders Association) with a MiniMentalState Examination (MMSE) score 25 and a Clinical Dementia Rating global score=0.5, 1, 2 or 3.

The terms “treating”, “treatment”, and the like are used herein to refer to obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment”, as used herein, covers any treatment of a disease in a mammal, particularly a human, and includes: (a) preventing the disease from occurring in a subject which may be predisposed to the disease but has not yet been diagnosed as having it, i.e., causing the clinical symptoms of the disease not to develop in a subject that may be predisposed to the disease but does not yet experience or display symptoms of the disease; (b) inhibiting the disease, i.e., arresting, reducing, or slowing the development of the disease or its clinical symptoms; (c) relieving the disease, i.e., causing regression of the disease and/or its symptoms or conditions.

A “therapeutically effective amount” means the amount of an active ingredient or combination of active ingredients that, when administered to a patient for treating a disease, is sufficient to effect such treatment for the disease. The “therapeutically effective amount” will vary depending on the disease and its severity and the age, weight, etc., of the mammal to be treated.

As used herein, the wording “a compound for use . . . ”, for example, shall be understood as being equivalent to the wording “use of a compound for . . . ” or “use of a compound for the preparation of a medicament for use in . . . ”.

For purposes of the present invention, references to 2-(cyclohexylmethyl)-N-{2-[(2S)-1-methylpyrrolidin-2-yl]ethyl}-1,2,3,4-tetrahydroisoquinoline-7-sulfonamide include the pharmaceutically acceptable salts, hydrates and solvates thereof, and solvates and hydrates of the pharmaceutically acceptable salts. In one aspect, the 2-(cyclohexylmethyl)-N-{2-[(2S)-1-methylpyrrolidin-2-yl]ethyl}-1,2,3,4-tetrahydroisoquinoline-7-sulfonamide is 2-(cyclohexylmethyl)-N-{2-[(2S)-1-methylpyrrolidin-2-yl]ethyl}-1,2,3,4-tetrahydroisoquinoline-7-sulfonamide difumarate monohydrate.

2-(Cyclohexylmethyl)-N-{2-[(2S)-1-methylpyrrolidin-2-yl]ethyl}-1,2,3,4-tetrahydroisoquinoline-7-sulfonamide, which has the structure of Formula (I):

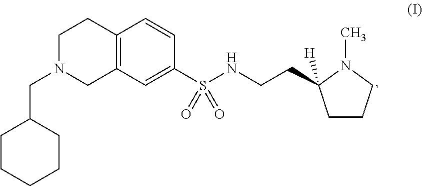 [SMILES: CN1CCC[C@H]1CCNS(=O)(=O)c1ccc2c(c1)CN(CC1CCCCC1)CC2]

is a potent, specific non-imidazole histamine H3 receptor (H3R) antagonist. A preparation and the physical properties and beneficial pharmacological properties of 2-(cyclohexylmethyl)-N-{2-[(2S)-1-methylpyrrolidin-2-yl]ethyl}-1,2,3,4-tetrahydroisoquinoline-7-sulfonamide are described in, for example, WO2005/118547 (also US2007/0105834). The difumarate monohydrate salt of 2-(cyclohexylmethyl)-N-{2-[(2S)-1-methylpyrrolidin-2-yl]ethyl}-1,2,3,4-tetrahydroisoquinoline-7-sulfonamide is described in WO2010/151611 (also US2012/0149728).

2-(Cyclohexylmethyl)-N-{2-[(2S)-1-methylpyrrolidin-2-yl]ethyl}-1,2,3,4-tetrahydroisoquinoline-7-sulfonamide improves cognitive performances on short-term and long-term memory as well as attention deficits in various rodent models, thus supporting the therapeutic potential of 2-(cyclohexylmethyl)-N-{2-[(2S)-1-methylpyrrolidin-2-yl]ethyl}-1,2,3,4-tetrahydroisoquinoline-7-sulfonamide for the symptomatic treatment of dementia of the Alzheimer's type (DAT) (Griebel, et al., Pharmacol Biochem Behay. (2012) 102(2), 203-14).

Cholinesterase inhibitors are thought to exert cognitive and functional improvement in Alzheimer's disease patients by increasing the concentration of acetylcholine through reversible inhibition of its hydrolysis by cholinesterases. Treatment with cholinesterase inhibitors, on average, delays the worsening of symptoms in about half of the patients who take them. The cholinesterase inhibitors currently approved in the U.S. are donepezil (Aricept®), rivastigmine (Exelon®), and galantamine (Reminyl®).

Applicants have now found that the co-administration of the histamine H3 receptor antagonist, 2-(cyclohexylmethyl)-N-{2-[(2S)-1-methylpyrrolidin-2-yl]ethyl}-1,2,3,4-tetrahydroisoquinoline-7-sulfonamide, with donepezil was significantly better than that of 2-(cyclohexylmethyl)-N-{2-[(2S)-1-methylpyrrolidin-2-yl]ethyl}-1,2,3,4-tetrahydroisoquinoline-7-sulfonamide, or donepezil administered individually in various disease assessments. More specifically, the superior benefit for a combination of a cholinesterase inhibitor and 2-(cyclohexylmethyl)-N-{2-[(2S)-1-methylpyrrolidin-2-yl]ethyl}-1,2,3,4-tetrahydroisoquinoline-7-sulfonamide has been demonstrated in two animal models of cognitive disruption and also on different measures of cognition, behavior and functional capabilities in a clinical trial with AD patients.

The present invention therefore provides a combination of 2-(cyclohexylmethyl)-N-{2-[(2S)-1-methylpyrrolidin-2-yl]ethyl}-1,2,3,4-tetrahydroisoquinoline-7-sulfonamide with a cholinesterase inhibitor which is more effective than a cholinesterase inhibitor or 2-(cyclohexylmethyl)-N-{2-[(2S)-1-methylpyrrolidin-2-yl]ethyl}-1,2,3,4-tetrahydroisoquinoline-7-sulfonamide alone.

In one aspect, the combination comprises administering to a patient in need thereof a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide in combination with a cholinesterase inhibitor.

Another aspect of the invention is the compound 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt thereof for use in combination with a cholinesterase inhibitor for the treatment of Alzheimer's disease in a patient.

Another aspect of the invention is the compound 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt thereof for use as an adjunct treatment in an Alzheimer's disease patient on stable treatment with a cholinesterase.

Another aspect of the invention is the compound 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt thereof as a safe and effective active ingredient for use in the adjunct treatment of Alzheimer's disease in a patient on stable treatment with a cholinesterase inhibitor.

Another aspect of the invention is a method of treating Alzheimer's disease in a patient comprising administering to said patient a therapeutically effective amount of a combination comprising 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof and a cholinesterase inhibitor.

Another aspect of the invention is a method for treating Alzheimer's disease in a patient comprising administering to said patient a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof and a therapeutically effective amount of a cholinesterase inhibitor.

In addition, this invention provides a method for the adjunctive treatment of Alzheimer's disease in a patient on stable treatment with a cholinesterase inhibitor, which comprises administering to said patient a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof.

The active ingredients comprising the combination or treatments of the present invention are administered repeatedly according to a protocol that depends on the patient to be treated (age, weight, treatment history, etc.), which can be determined by a skilled physician.

In some aspects of the invention, the 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof is 2-(cyclohexylmethyl)-N-{2-[(2S)-1-methylpyrrolidin-2-yl]ethyl}-1,2,3,4-tetrahydroisoquinoline-7-sulfonamide difumarate monohydrate.

In some aspects of the invention, the cholinesterase inhibitor is selected from the group consisting of galantamine, rivastigmine, donepezil, mimopezil, ladostigil, and huperzine A; or a pharmaceutically acceptable salt thereof. In some aspects of the invention, the cholinesterase inhibitor is selected from the group consisting of galantamine, rivastigmine, and donepezil; or a pharmaceutically acceptable salt thereof. In some aspects of the invention, the cholinesterase inhibitor is donepezil or a pharmaceutically acceptable salt thereof. In a particular aspect, the donepezil is donepezil hydrochloride.

In another aspect, the treatment comprises administering to the patient about 5 mg to about 10 mg of donepezil per day.

In another aspect, the treatment comprises administering to the patient a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide, or pharmaceutically acceptable salt thereof, and cholinesterase inhibitor, with respect to the individual components.

Another aspect of the invention is a method for treating Alzheimer's disease in a patient comprising administering to said patient a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof.

In some embodiments of the invention, the treatment of Alzheimer's disease comprises treatment of one or more symptoms of Alzheimer's disease. For example, the treatment of Alzheimer's disease comprises treatment of one or more symptoms selected from the group consisting of disturbances of memory, disturbances of praxis, disturbances of attention, confusion, irritability and aggression, mood swings, language breakdown, long-term memory loss, withdrawal of the sufferer, loss of motor control, impairment of (cognitive) executive functions, functional disability, and apathy.

In some embodiments of the invention, the treatment comprises suppression of the progression of the symptoms of dementia.

In some embodiments of the invention, the treatment comprises reduction in the progression of the symptoms of dementia.

In another aspect, the treatment of Alzheimer's disease comprises an improvement in one or more factors selected from the group consisting of the maintenance or improvement of cognition (which can be measured by the ADAS-Cog subscale); maintenance or improvement of Activities of Daily Living (which can be measured by ADCS-ADL subscale); maintenance or improvement of one or more factors from the Cognitive Drug Research System (CDR-S) computerized assessment; maintenance or improvement of the Apathy Evaluation Scale-Informant (AES-I) score; and maintenance or improvement of CGIC.

In some embodiments, the treatment comprises a reduction in the worsening of cognitive function of the patient, which can be measured by the ADAS-Cog.

In some embodiments, the treatment comprises a reduction in the worsening of functional capability of the patient, which can be measured by the ADCS-ADL.

In some embodiments, the treatment comprises a reduction in the worsening of attention of the patient, which can be measured by the CDR-S power of attention.

In some embodiments, the treatment comprises a maintenance or reduction in the symptoms of apathy of the patient, which can be measured by the AES-I.

In some embodiments, the treatment comprises an increase in patient responder rate based on a combined criterion, which is measured with ADAS-Cog and ADCS-ADL and CGIC.

In some embodiments, the treatment comprises a maintenance in a sleep parameter selected from total sleep time, number of awakenings, interdaily variability and intradaily stability; which parameters can be measured by Actigraphy.

In some embodiments, the treatment comprises an alteration in sleep parameters selected from sleep fragmentation, sleep efficiency, wake after sleep onset, mean awakening duration and relative amplitude; which parameters can be measured by Actigraphy.

In some embodiments, the treatment comprises an increase in episodic memory of the patient, which can be measured by the CDR-S.

In some embodiments of the invention, the patient has dementia of the Alzheimer's type (DAT).

In some embodiments of the invention, the patient has mild Alzheimer's disease.

In some embodiments of the invention, the patient has moderate Alzheimer's disease.

In other embodiments of the invention, the patient has preclinical Alzheimer's disease.

In other embodiments of the invention, the patient has mild cognitive impairment due to Alzheimer's disease.

In one aspect, the treatment comprises administering to the patient about 0.10 to about 20 mg of 2-(cyclohexylmethyl)-N-{2-[(2S)-1-methylpyrrolidin-2-yl]ethyl}-1,2,3,4-tetrahydroisoquinoline-7-sulfonamide, or pharmaceutically acceptable salt (measured in base form) thereof, per day. In a particular aspect, the treatment comprises administering to the patient about 0.5 to about 5 mg (about 0.5 mg, about 1 mg, about 2 mg, about 3 mg, about 4 mg, or about 5 mg, for example) of 2-(cyclohexylmethyl)-N-{2-[(2S)-1-methylpyrrolidin-2-yl]ethyl}-1,2,3,4-tetrahydroisoquinoline-7-sulfonamide, or pharmaceutically acceptable salt (measured in base form) thereof, per day. In another particular aspect, the treatment comprises administering to the patient about 5 to about 10 mg of 2-(cyclohexylmethyl)-N-{2-[(2S)-1-methylpyrrolidin-2-yl]ethyl}-1,2,3,4-tetrahydroisoquinoline-7-sulfonamide, or pharmaceutically acceptable salt (measured in base form) thereof, per day. In another particular aspect, the treatment comprises administering to the patient about 5 mg of 2-(cyclohexylmethyl)-N-{2-[(2S)-1-methylpyrrolidin-2-yl]ethyl}-1,2,3,4-tetrahydroisoquinoline-7-sulfonamide, or pharmaceutically acceptable salt (measured in base form) thereof, per day.

The active ingredients of the present invention can be administered simultaneously or separately over time. For example, the cholinesterase inhibitor may be taken once daily in the evening, while the 2-(cyclohexylmethyl)-N-{2-[(2S)-1-methylpyrrolidin-2-yl]ethyl}-1,2,3,4-tetrahydroisoquinoline-7-sulfonamide may be administered once daily in the morning. However, simultaneous administration of 2-(cyclohexylmethyl)-N-{2-[(2S)-1-methylpyrrolidin-2-yl]ethyl}-1,2,3,4-tetrahydroisoquinoline-7-sulfonamide and cholinesterase inhibitor is within the scope of the invention. Accordingly, in one aspect of the invention, the 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof is administered simultaneously with the cholinesterase inhibitor.

Modes of administration for each active ingredient include, but are not limited to, oral, parenteral (e.g., subcutaneous, subdural, intravenous, intramuscular, intrathecal, intraperitoneal, intracerebral, intraarterial, or intralesional routes of administration), topical, localized (e.g., surgical application or surgical suppository), rectal, and pulmonary (e.g., aerosols, inhalation, or powder). The route of administration would be based on the composition being administered, tissue targeting, and the like, as would be known to the artisan of ordinary skill. The route of administration may be varied in any way, limited by the physical properties of the active ingredients and the convenience of the patient and the caregiver.

The combinations or therapies of the present invention do not require that the active ingredients are administered by the same route. For example, a composition comprising one active ingredient can be administered orally while a composition comprising the other active ingredient is administered transdermally.

The drugs or active ingredients comprising the combination or therapy of the present invention are usually administered in the form of pharmaceutical compositions. The pharmaceutical compositions contain one or more active ingredients associated with one or more pharmaceutically acceptable carriers or excipients.

The excipient employed is typically one suitable for administration to human subjects or other mammals. Pharmaceutically acceptable excipients or carriers include at least one component selected from the group comprising pharmaceutically acceptable carriers, diluents, coatings, adjuvants, excipients, or vehicles, such as preserving agents, fillers, disintegrating agents, wetting agents, emulsifying agents, stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms.

The pharmaceutical compositions can be prepared using conventional techniques known to those skilled in the art.

By way of example, a unit administration form of 2-(cyclohexylmethyl)-N-{2-[(2S)-1-methylpyrrolidin-2-yl]ethyl}-1,2,3,4-tetrahydroisoquinoline-7-sulfonamide difumarate monohydrate is as follows:

| | |
|---|---|
| 2-(Cyclohexylmethyl)-N-{2-[(2S)-1-methylpyrrolidin-2- | 5 mg |
| yl]ethyl}-1,2,3,4-tetrahydroisoquinoline-7-sulfonamide | |
| difumarate monohydrate | |
| Lactose monohydrate | 185.0 mg |
| Microcrystalline cellulose | 37.5 mg |
| Hydroxypropyl methylcellulose (6 mPa.s) | 10.0 mg |
| Croscarmellose sodium | 10.0 mg |
| Magnesium stearate | 2.5 mg |

The actual amount of each active ingredient in the combinations or therapies according to the present invention will depend on a number of factors, such as the severity of the disease, i.e., the condition or disease to be treated, age and relative health of the subject, the potency of the compound used, the route and form of administration, and other factors.

The following examples will further illustrate the invention, without, however, limiting it thereto.

### EXAMPLE 1

### Preclinical Studies

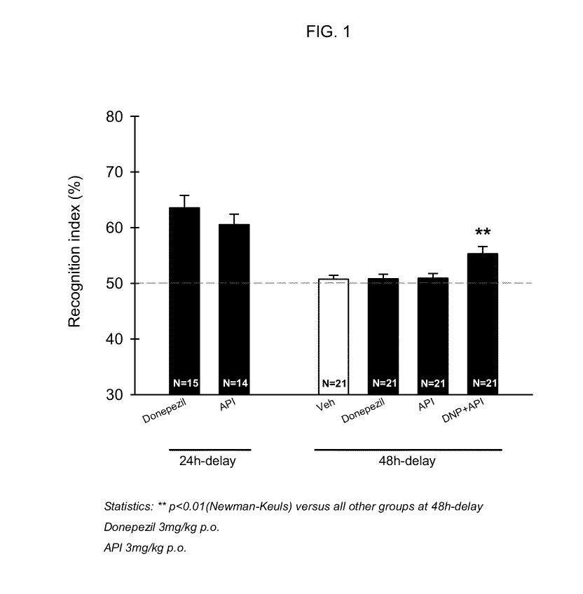

In an object recognition test, oral administration of 2-(cyclohexylmethyl)-N-{2-[(2S)-1-methylpyrrolidin-2-yl]ethyl}-1,2,3,4-tetrahydroisoquinoline-7-sulfonamide difumarate monohydrate (3 mg/kg) or donepezil hydrochloride (DNP) (3 mg/kg) individually enhanced the memory of CD1 male mice 24 hours after administration, contrasting with lack of procognitive effect observed for either molecule after 48 hours. However, co-administration of 3 mg/kg of 2-(cyclohexylmethyl)-N-{2-[(2S)-1-methylpyrrolidin-2-yl]ethyl}-1,2,3,4-tetrahydroisoquinoline-7-sulfonamide difumarate monohydrate with 3 mg/kg donepezil hydrochloride had a significant procognitive effect 48 hours post administration. This procognitive effect of co-administration at 48 hours suggests a potentiating effect of 2-(cyclohexylmethyl)-N-{2-[(2S)-1-methylpyrrolidin-2-yl]ethyl}-1,2,3,4-tetrahydroisoquinoline-7-sulfonamide difumarate monohydrate on the procognitive effect of donepezil hydrochloride. The results of this study are depicted in FIG. 1.

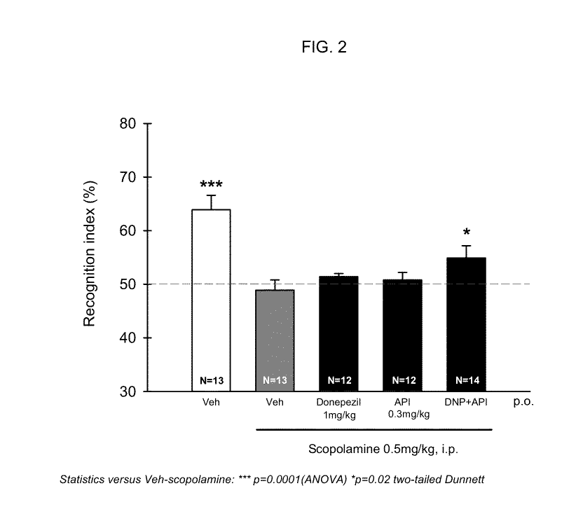

In another study, co-treatment of inactive amounts of 2-(cyclohexylmethyl)-N-{2-[(2S)-1-methylpyrrolidin-2-yl]ethyl}-1,2,3,4-tetrahydroisoquinoline-7-sulfonamide difumarate monohydrate (0.3 mg/kg) with donepezil hydrochloride (1 mg/kg) in scopolamine treated mice reversed episodic memory deficits suggesting a beneficial effect of a combination of these two drugs on cognition. The results of this study are depicted in FIG. 2.

### EXAMPLE 2

### Clinical Study

A randomized, double-blind, parallel-group, placebo-controlled clinical trial was performed to study the effect on cognitive performance, safety, and tolerability of 2-(cyclohexylmethyl)-N-{2-[(2S)-1-methylpyrrolidin-2-yl]ethyl}-1,2,3,4-tetrahydroisoquinoline-7-sulfonamide difumarate monohydrate as adjunct treatment to donepezil.

More specifically, patients with mild to moderate Alzheimer's disease on stable donepezil therapy were randomized to receive doses of 0.5 mg, 2 mg, or 5 mg/day of 2-(cyclohexylmethyl)-N-{2-[(2S)-1-methylpyrrolidin-2-yl]ethyl}-1,2,3,4-tetrahydroisoquinoline-7-sulfonamide difumarate monohydrate or placebo for 24 weeks, with a 10 week follow-up period.

291 patients were enrolled (290 treated) in the study. The baseline characteristics of the randomized patients are described in Table 1:

TABLE 1
Randomized Population

| | Placebo | 0.5 mg | 2 mg | 5 mg | TOTAL |
|---|---|---|---|---|---|
| | (N = 78) | (N = 72) | (N = 72) | (N = 69) | (N = 291) |
| Age, y (SD) | 72.6 (6.8) | 71.7 (7.5) | 73.8 (7.8) | 72.2 (7.9) | 72.6 (7.9) |
| Gender: female | 36 (46.2) | 37 (51.4) | 31 (43.1) | 36 (52.2) | 140 (48.1) |
| No. (%) | | | | | |
| Race: Caucasian | 77 (98.7) | 71 (98.6) | 72 (100) | 68 (98.6) | 288 (99.0) |
| No. (%) | | | | | |
| MMSE | 20.5 (2.9) | 20.6 (3.2) | 20.8 (2.4) | 21.7 (2.6) | 20.9 (2.8) |
| total score (SD) | | | | | |
| Mild, No (%) | 49 (62.8%) | 44 (61.1%) | 52 (72.2%) | 57 (82.6%) | 202 (69.4%) |
| Moderate, No (%) | 29 (37.2%) | 28 (38.9%) | 20 (27.8%) | 12 (17.4%) | 89 (30.6%) |
| ADAS-Cog 11 | 24.1 (9.6) | 19.9 (7.0) | 21.5 (6.6) | 20.6 (6.6) | 21.6 (7.8) |
| total score (SD) | | | | | |
| ADCS-ADL | 60.1 (12.2) | 61.5 (10.8) | 60.8 (11.1) | 62.7 (10.6) | 61.2 (11.2) |
| total score (SD) | | | | | |

The Modified Intent-to-Treat (mITT) population are all randomized patients who received at least one dose of 2-(cyclohexylmethyl)-N-{2-[(2S)-1-methylpyrrolidin-2-yl]ethyl}-1,2,3,4-tetrahydroisoquinoline-7-sulfonamide difumarate monohydrate with available ADAS-Cog standard 11-items total score at baseline and at least one post-baseline assessment.

The patient recruitment was carried out by taking into account the following inclusion criteria:
1. Outpatients with diagnosis of AD based on the Dementia of Alzheimer Type (DAT) Diagnostic and Statistical Manual for Mental Disorders, 4^{th} edition (DSM-4) criteria and the National Institute of Neurological and Communicative Disorders and Stroke/Alzheimer's Disease and Related Disorders Association (NINCDS/ADRDA) criteria for probably AD.
   The diagnosis should also be supported by a Modified Hachinski ≦4 score and a magnetic resonance imaging (MRI) or computed tomography (CT)-scan performed within 12 months prior to randomization that is compatible with the diagnosis of AD and should not reveal other causes of dementia.
2. During screening, the mild to moderate range of severity should be established by Mini Mental State Examination (MMSE) score ≧15 and ≦25 as well Clinical Dementia Rating (CDR) global score should be 0.5, 1, or 2.
3. Patient is under stable and well-tolerated donepezil treatment at a fixed dose of 5 or 10 mg daily for at least 3 months prior to screening visit; and
4. Patient is ≧55 years old.

II. Duration, Treatment and Results

Patients were randomized to receive 0.5 mg, 2 mg, or 5 mg of 2-(cyclohexylmethyl)-N-{2-[(2S)-1-methylpyrrolidin-2-yl]ethyl}-1,2,3,4-tetrahydroisoquinoline-7-sulfonamide difumarate monohydrate (measured in base form), or matching placebo once a day, with donepezil (5 mg or 10 mg in the form the patient took prior to enrollment), for 24 weeks, with or without food.

The results were evaluated according to the following criteria:

Change from baseline to Week 24 in the standard 11-item total score from the 13-item Alzheimer's Disease Cooperative Study—Cognitive subscale (ADAS-Cog);

Change from baseline to Week 24 in the Alzheimer's Disease Cooperative Study-Activities of Daily Living (ADCS-ADL) Inventory global score (which assesses basic and instrumental ADL, through 23 standardized questions to a caregiver);

Change from baseline to Week 24 in the five factors from the Cognitive Drug Research computerized assessment system (CDR-S): power of attention, continuity of attention, quality of working memory, cognitive reaction time, and speed of memory;

Change from baseline to Week 8 and 24 in the Cognitive Drug Research computerized assessment system (CDR-S) episodic memory;

Change from baseline to Week 12 and 24 in the Apathy Evaluation Scale for Informant (AES-I) score; and

Change from baseline to Week 24 in the Neuropsychiatric Inventory (NPI) domain scores of irritability/lability, anxiety, agitation/aggression, sleep and nighttime behavior disorders, aberrant motor behaviour, apathy/indifference, appetite/eating change, delusions, depression/dysphoria, disinhibition, elation/euphoria, and hallucinations.

Significant improvements were found for the 5 mg group versus placebo on 1) the ADAS-Cog11 total score change from baseline at week 34 (follow-up (FUP) after 10 weeks of treatment discontinuation), p=0.048, 2) ADCS-ADL total score change from baseline at week 24, p=0.02, 3) Apathy (AES-I) total score change from baseline at week 12, p=0.04, and 4) Episodic memory score change from baseline at week 8, p=0.02. A difference in the rate of responder on a combined criterion considering ADAS-Cog, ADCS-ADL and CGIC scores, at week 24 was observed in favor of the 5 mg group (16.7%) versus placebo (3.9%); p=0.01.

A tendency (NS, p<0.2) for improvement of attentional capabilities (power of attention) was shown with CDR-S change from baseline at week 24 (5 and also 0.5 mg doses). The other measures of attention/speed of response were not significantly modified at week 24, neither was the measure of working memory.

No relevant modification was observed on the domains of the NPI. The NPI results are presented in Table 2.

TABLE 2

| | | Placebo | 0.5 mg | 2 mg | 5 mg |
|---|---|---|---|---|---|
| Irritability | baseline | 1.13 | 1.19 | 0.90 | 1.00 |
| lability | Ch from bl W12 | 0.28 | −0.07 | 0.47 | 0.03 |
| | Ch from bl W24 | 0.45 | 0.09 | 0.63 | 0.47 |
| | Ch from bl to | | NS | NS | NS |
| | W24 vs placebo | | | | |
| Anxiety | baseline | 1.10 | 1.19 | 0.84 | 1.00 |
| | Ch from bl W12 | 0.03 | 0.20 | −0.03 | 0.12 |
| | Ch from bl W24 | 0.35 | 0.16 | 0.15 | 0.12 |
| | Ch from bl to | | NS | NS | NS |
| | W24 vs placebo | | | | |
| Agitation/ | baseline | 1.06 | 1.10 | 0.52 | 1.02 |
| Aggression | Ch from bl W12 | 0.10 | 0.23 | 0.48 | −0.12 |
| | Ch from bl W24 | 0.31 | 0.04 | 0.68 | 0.58 |
| | Ch from bl to | | NS | NS | NS |
| | W24 vs placebo | | | | |
| Sleep and | baseline | 0.67 | 0.86 | 0.87 | 0.64 |
| nighttime | Ch from bl W12 | 0.44 | 0.30 | 0.33 | 0.08 |
| behavior | Ch from bl W24 | 0.41 | −0.23 | 0.19 | 0.71 |
| | Ch from bl to | | NS | NS | NS |
| | W24 vs placebo | | | | |
| Abberant motor | baseline | 0.56 | 0.67 | 0.50 | 1.25 |
| behavior | Ch from bl W12 | 0.26 | −0.08 | 0.25 | −0.11 |
| | Ch from bl W24 | 0.55 | 0.42 | 0.56 | −0.69 |
| | Ch from bl to | | NS | NS | p = 0.04 |
| | W24 vs placebo | | | | |
| Apathy/ | baseline | 2.19 | 2.29 | 1.46 | 2.18 |
| Indifference | Ch from bl W12 | 0.56 | −0.21 | 0.26 | 0.09 |
| | Ch from bl W24 | 0.56 | 0.13 | 0.92 | 0.10 |
| | Ch from bl to | | NS | NS | NS |
| | W24 vs placebo | | | | |
| Appetite/Eating | baseline | 0.93 | 1.34 | 0.58 | 1.37 |
| changes | Ch from bl W12 | 0.04 | −0.58 | −0.18 | −0.17 |
| | Ch from bl W24 | 0.08 | −0.33 | −0.02 | 0.1 |
| | Ch from bl to | | NS | NS | NS |
| | W24 vs placebo | | | | |
| Delusions | baseline | 0.30 | 0.08 | 0.22 | 0.31 |
| | Ch from bl W12 | 0.12 | 0.31 | −0.05 | 0.00 |
| | Ch from bl W24 | 0.32 | 0.28 | 0.21 | 0.04 |
| | Ch from bl to | | NS | NS | NS |
| | W24 vs placebo | | | | |
| Depression/ | baseline | 1.21 | 1.16 | 0.78 | 0.86 |
| Dysphoria | Ch from bl W12 | −0.01 | 0.00 | 0.35 | −0.07 |
| | Ch from bl W24 | −0.12 | 0.07 | 0.12 | 0.25 |
| | Ch from bl to | | NS | NS | NS |
| | W24 vs placebo | | | | |
| Disinhibition | baseline | 0.59 | 0.69 | 0.19 | 0.71 |
| | Ch from bl W12 | −0.04 | −0.13 | 0.08 | 0.00 |
| | Ch from bl W24 | 0.11 | −0.06 | 0.03 | 0.06 |
| | Ch from bl to | | NS | NS | NS |
| | W24 vs placebo | | | | |
| Elation/ | baseline | 0.36 | 0.24 | 0.19 | 0.05 |
| Euphoria | Ch from bl W12 | −0.15 | 0.15 | −0.06 | 0.14 |
| | Ch from bl W24 | 0.00 | 0.20 | 0.07 | 0.10 |
| | Ch from bl to | | NS | NS | NS |
| | W24 vs placebo | | | | |
| Hallucination | baseline | 0.03 | 0.08 | 0.10 | 0.28 |
| | Ch from bl W12 | 0.17 | 0.05 | 0.02 | −0.14 |
| | Ch from bl W24 | 0.02 | −0.06 | 0.02 | −0.15 |
| | Ch from bl to | | NS | NS | NS |
| | W24 vs placebo | | | | |

Ch = change;
bl = baseline;
W12 = week 12;
W24 = week 24;
NS = non-significant

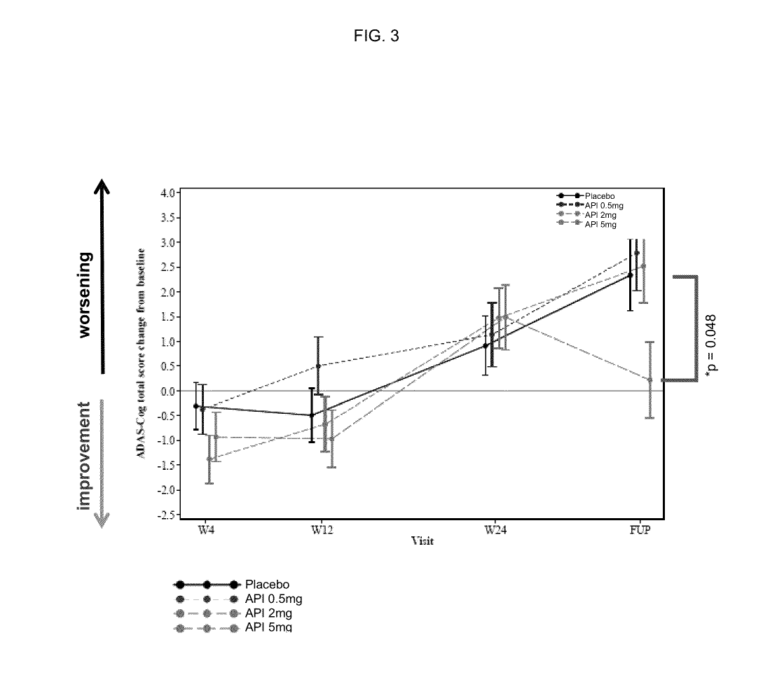

FIG. 3 illustrates the time course for the change from baseline in ADAS-Cog scores for all four dose groups through follow-up in the mITT population. After 24 weeks of treatment, the mean differences in the ADAS-Cog change score for 2-(cyclohexylmethyl)-N-{2-[(2S)-1-methylpyrrolidin-2-yl]ethyl}-1,2,3,4-tetrahydroisoquinoline-7-sulfonamide difumarate monohydrate treated patients were not significantly different. At follow-up, however, there was a statistically significant improvement in the ADAS-Cog score in patients on the 5 mg/day dose when compared to placebo.

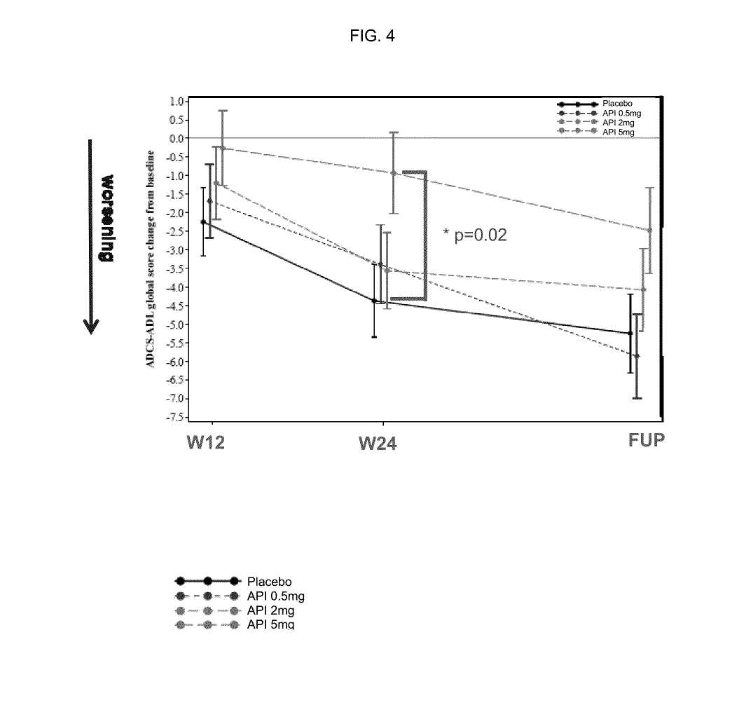

FIG. 4 illustrates the time course for the change from baseline in ADCS-ADL scores for all four dose groups through follow-up in the mITT population. After 24 weeks of treatment, there was a significant difference between the patients treated with 5 mg/day of 2-(cyclohexylmethyl)-N-{2-[(2S)-1-methylpyrrolidin-2-yl]ethyl}-1,2,3,4-tetrahydroisoquinoline-7-sulfonamide difumarate monohydrate and placebo, which suggests a smaller decline in functional capabilities in this patient group. At follow-up, the trend in slope was similar for all dose groups.

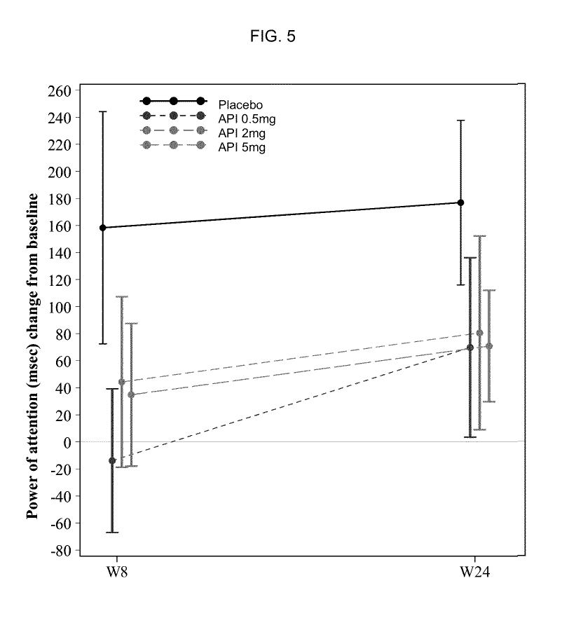

FIG. 5 illustrates the time course for the change from baseline in power of attention (in msec) for all four dose groups through week 24 in the mITT population. There was no significant difference between groups of patients treated with 2-(cyclohexylmethyl)-N-{2-[(2S)-1-methylpyrrolidin-2-yl]ethyl}-1,2,3,4-tetrahydroisoquinoline-7-sulfonamide difumarate monohydrate and placebo, either at week 8 or week 24. Tendencies for improvement versus placebo were seen for 0.5 and 5 mg group at week 24 suggesting a smaller decline in attention capabilities in these patient groups.

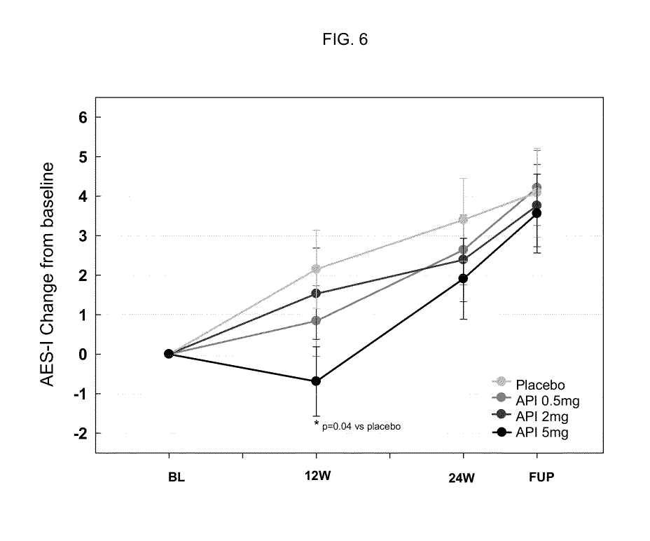

FIG. 6 illustrates the time course for the change from baseline in AES-I scores for all four dose groups through follow-up in the mITT population. After 12 weeks of treatment, there was a significant difference between the patients treated with 5 mg/day of 2-(cyclohexylmethyl)-N-{2-[(2S)-1-methylpyrrolidin-2-yl]ethyl}-1,2,3,4-tetrahydroisoquinoline-7-sulfonamide difumarate monohydrate and placebo, which demonstrates an improvement in apathy in this patient group. At 24 weeks and follow-up, the difference is no longer significant indicating transient symptomatic benefit of 2-(cyclohexylmethyl)-N-{2-[(2S)-1-methylpyrrolidin-2-yl]ethyl}-1,2,3,4-tetrahydroisoquinoline-7-sulfonamide difumarate monohydrate on apathy.

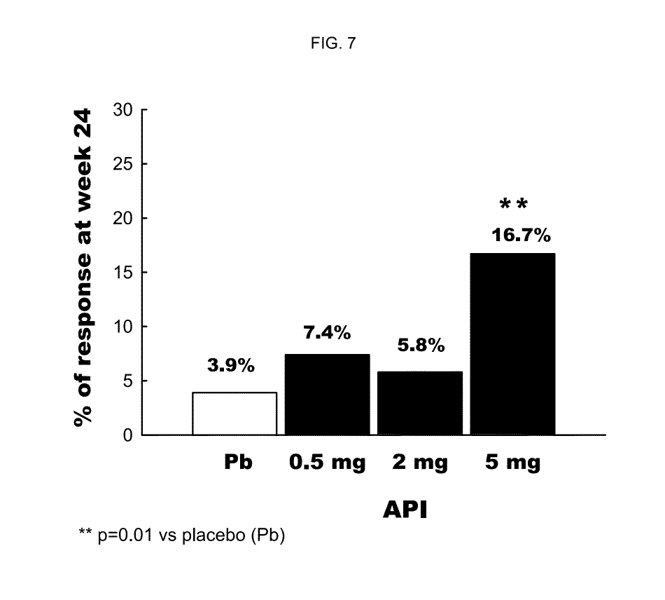

FIG. 7 illustrates the responder analysis at week 24 based on a combined criterion defined as followed:

change from baseline to Week 24 lower than or equal to −2 points in the ADAS-Cog total score, and

change from baseline to Week 24 greater than or equal to 0 point in the ADCS-ADL global score, and

no worsening according to the CGIC assessment at Week 24.

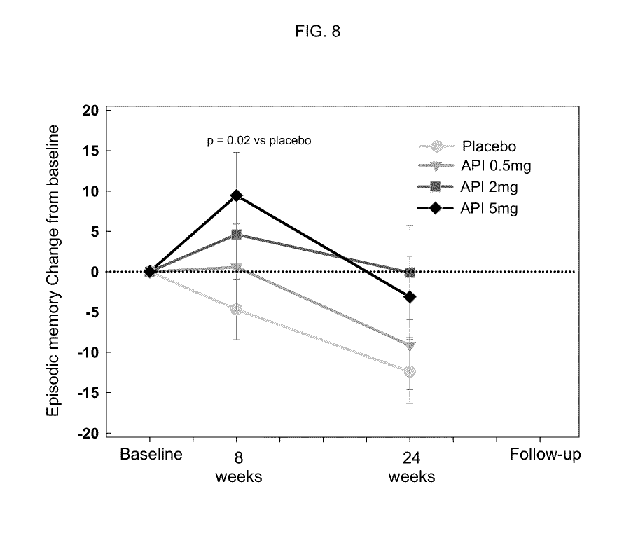

FIG. 8 illustrates the time course for the change from baseline in episodic memory for all four dose groups through week 24 in the mITT population. After 8 weeks of treatment, there was a significant difference between the patients treated with 5 mg/day of 2-(cyclohexylmethyl)-N-{2-[(2S)-1-methylpyrrolidin-2-yl]ethyl}-1,2,3,4-tetrahydroisoquinoline-7-sulfonamide difumarate monohydrate and placebo, which demonstrates an improvement in episodic memory in this patient group. At 24 weeks, the difference is no longer significant indicating transient symptomatic benefit of 2-(cyclohexylmethyl)-N-{2-[(2S)-1-methylpyrrolidin-2-yl]ethyl}-1,2,3,4-tetrahydroisoquinoline-7-sulfonamide difumarate monohydrate on episodic memory.

A difference is observed between the patients treated with 5 mg/day of 2-(cyclohexylmethyl)-N-{2-[(2S)-1-methylpyrrolidin-2-yl]ethyl}-1,2,3,4-tetrahydroisoquinoline-7-sulfonamide difumarate monohydrate over placebo as indicated by the higher rate of “responders” in this patient group. This means a greater proportion of patients treated with 5 mg dose show a stabilization or improvement of key measures of AD over 6 months.

2-(Cyclohexylmethyl)-N-{2-[(2S)-1-methylpyrrolidin-2-yl]ethyl}-1,2,3,4-tetrahydroisoquinoline-7-sulfonamide difumarate monohydrate was found to be safe and well tolerated. Table 3 describes the incidence of treatment emergent adverse events (TEAE).

TABLE 3

| | API | | | |
|---|---|---|---|---|
| | Placebo | 0.5 mg | 2 mg | 5 mg |
| | (N = 78) | (N = 72) | (N = 72) | (N = 69) |
| Patients with an TEAE | 61 (78.2%) | 53 (73.6%) | 54 (76.1%) | 59 (85.5%) |
| Patients with any treatment | 12 (15.4%) | 10 (13.9%) | 10 (14.1%) | 8 (11.6%) |
| emergent serious adverse | | | | |
| event | | | | |
| Infections and infestations | 23 (29.5%) | 20 (27.8%) | 11 (15.5%) | 16 (23.2%) |
| Psychiatric disorders | 20 (25.6%) | 21 (29.2%) | 18 (25.4%) | 27 (39.1%) |
| Anxiety | 3 (3.8%) | 3 (4.2%) | 4 (5.6%) | 4 (5.8%) |
| Depressive disorders | 1 (1.3%) | 1 (1.4%) | 2 (2.8%) | 5 (7.2%) |
| Sleep disorders and | 8 (10.3%) | 12 (16.7%) | 11 (15.5%) | 21 (30.4%) |
| Disturbance | | | | |
| Cardiac disorders | 8 (10.3%) | 5 (6.9%) | 3 (4.2%) | 5 (7.2%) |
| Vascular disorders | 12 (15.4%) | 2 (2.8%) | 3 (4.3%) | 3 (4.3%) |
| Gastrointestinal disorders | 13 (16.7%) | 11 (15.3%) | 18 (25.4%) | 17 (24.6%) |
| Diarrhea | 4 (5.1%) | 7 (9.7%) | 7 (9.9%) | 4 (5.8%) |
| Dyspepsia | 0 | 1 (1.4%) | 1 (1.4%) | 3 (4.3%) |
| Hiatus hernia | 0 | 0 | 0 | 3 (4.3%) |
| Skin and subcutaneous tissue | 6 (7.7%) | 7 (9.7%) | 15 (21.1%) | 5 (7.2%) |
| disorders | | | | |
| Dermatitis | 1 (1.3%) | 1 (1.4%) | 3 (4.2%) | 3 (4.3%) |
| Rashes | 1 (1.3%) | 1 (1.4%) | 4 (5.6%) | 1 (1.4%) |
| Musculoskeletal and | 6 (7.7%) | 5 (6.9%) | 12 (16.9%) | 16 (23.2%) |
| connective tissue disorders | | | | |
| Muscle spasm | 0 | 1 (1.4%) | 2 (2.8%) | 7 (10.6%) |
| Other (Falls) | 5 (6.4%) | 9 (12.5%) | 7 (9.9%) | 7 (10.1%) |
| Lens opacification (confirmed | 0 | 1/68 (1.5%) | 1/61 (1.6%) | 3/63 (4.3%) |
| Class II grade events) | | | | |

Sleep disorders and disturbances were the most prominent adverse events reported in the trial with the highest incidence in the group treated with 5 mg of 2-(cyclohexylmethyl)-N-{2-[(25)-1-methylpyrrolidin-2-yl]ethyl}-1,2,3,4-tetrahydroisoquinoline-7-sulfonamide difumarate monohydrate. This observation is consistent with the mechanism of action of H3 receptor antagonists which involves releasing histamine and acetylcholine, the main actors of waking states.

A low incidence in vascular disorders were reported for all groups treated with 2-(cyclohexylmethyl)-N-{2-[(2S)-1-methylpyrrolidin-2-yl]ethyl}-1,2,3,4-tetrahydroisoquinoline-7-sulfonamide difumarate monohydrate over placebo.

Table 4 describes the actigraphy parameters.

TABLE 4

| | | Placebo | 0.5 mg | 2 mg | 5 mg |
|---|---|---|---|---|---|
| Sleep | baseline | 33.16 | 33.11 | 37.58 | 35.02 |
| fragmentation | Ch from bl W2 | 1.51 | 0.91 | 2.82 | 3.57 |
| | Ch from bl W12 | −0.03 | 0.29 | 6.31 | 5.83 |
| | | | NS | <.0001 | 0.0005 |
| | Ch from bl FUP | 0.35 | 0.98 | 3.93 | 3.08 |
| Sleep | baseline in % | 82.63 | 83.96 | 83.25 | 83.09 |
| efficiency | Ch from bl W2 | 0.59 | −0.05 | −1.65 | −1.82 |
| | Ch from bl W12 | 2.38 | −0.53 | −2.58 | −2.26 |
| | | | 0.0121 | <.0001 | <.0001 |
| | Ch from bl FUP | −0.28 | −0.69 | −3.97 | −0.89 |
| Wake after | baseline in h.min | 1.00 | 0.55 | 1.00 | 1.00 |
| sleep onset | Ch from bl W2 | −0.01 | 0.02 | 0.07 | 0.08 |
| | Ch from bl W12 | −0.03 | 0.03 | 0.18 | 0.17 |
| | | | NS | <.0001 | <.0001 |
| | Ch from bl FUP | 0.02 | 0.04 | 0.13 | 0.09 |
| Mean | baseline in min | 2.43 | 2.37 | 2.43 | 2.36 |
| awakening | Ch from bl W2 | 0.02 | 0.08 | 0.14 | 0.30 |
| duration | Ch from bl W12 | −0.03 | 0.22 | 0.40 | 0.38 |
| | | | 0.0109 | <.0001 | <.0001 |
| | Ch from bl FUP | 0.02 | 0.01 | 0.34 | 0.12 |
| Relative | baseline | 0.87 | 0.84 | 0.85 | 0.87 |
| amplitude | Ch from bl W2 | 0.00 | 0.00 | −0.03 | −0.02 |
| | Ch from bl W12 | −0.01 | 0.02 | −0.06 | −0.08 |
| | | | NS | <.0001 | 0.0015 |
| | Ch from bl FUP | −0.01 | −0.01 | −0.07 | −0.02 |
| Total sleep | baseline in h.min | 7.4 | 7.36 | 7.43 | 7.22 |
| time | Ch from bl W2 | 0.02 | −0.03 | −0.1 | −0.11 |
| | Ch from bl W12 | 0.03 | 0.05 | −0.2 | −0.05 |
| | | | NS | NS (p = | NS |
| | | | | 0.06) | |
| | Ch from bl FUP | 0.1 | 0.24 | −0.25 | 0.1 |
| Number of | baseline | 22.35 | 21.53 | 22.72 | 22.82 |
| awakenings | Ch from bl W2 | −0.31 | −0.37 | 0.51 | −0.52 |
| | Ch from bl W12 | 0.17 | −0.55 | 1.26 | 0.84 |
| | | | NS | NS | NS |
| | Ch from bl FUP | −0.30 | 1.34 | 1.27 | 1.20 |
| Interdaily | baseline | 0.55 | 0.48 | 0.50 | 0.54 |
| variability | Ch from bl W2 | 0.00 | 0.01 | −0.01 | 0.00 |
| | Ch from bl W12 | −0.02 | 0.04 | −0.03 | −0.04 |
| | | | NS | NS | NS |
| | Ch from bl FUP | 0.00 | −0.02 | −0.01 | 0.00 |
| Intradaily | baseline | 0.94 | 0.97 | 1.01 | 0.92 |
| stability | Ch from bl W2 | 0.01 | −0.03 | 0.02 | −0.05 |
| | Ch from bl W12 | 0.02 | −0.06 | 0.01 | 0.02 |
| | | | NS | NS | NS |
| | Ch from bl FUP | 0.26 | 0.06 | 0.04 | −0.03 |

Ch = change;
bl = baseline;
FUP = follow-up;
W2 = week 2;
W12 = week 12;
NS = non-significant

Activity measured by the Actiwatch system showed disruption during the nighttime period for all groups at baseline and a dose-dependent worsening of sleep quality from week 12, as assessed by the increase in wake after sleep onset (WASO) in groups treated with 2 mg and 5 mg of 2-(cyclohexylmethyl)-N-{2-[(2S)-1-methylpyrrolidin-2-yl]ethyl}-1,2,3,4-tetrahydroisoquinoline-7-sulfonamide difumarate monohydrate over placebo. No modification of total sleep time was seen at the dose of 5 mg suggesting a potential for compensation by increasing the time spent asleep. After treatment discontinuation (FUP measures), total sleep time and WASO improved for the group treated with 5 mg of 2-(cyclohexylmethyl)-N-{2-[(2S)-1-methylpyrrolidin-2-yl]ethyl}-1,2,3,4-tetrahydroisoquinoline-7-sulfonamide difumarate monohydrate which indicated potential for recovery.

1. A method of treating Alzheimer's disease comprising administering to a patient in need thereof a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide or a pharmaceutically acceptable salt thereof in combination with a cholinesterase inhibitor.

2.-3. (canceled)

4. The method according to claim 1, wherein the patient has mild Alzheimer's disease.

5. The method according claim 1, wherein the patient has moderate Alzheimer's disease.

6. The method according to claim 1, wherein the progression of the symptoms of dementia are suppressed.

7. The method according to claim 1, wherein about 5 mg per day of 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof is administered to the patient.

8. (canceled)

9. The method according to claim 1, wherein the 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof and cholinesterase inhibitor are each administered to the patient in a therapeutically effective amount with respect to the individual components.

10. The method according to claim 1, wherein the 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof is 2-(cyclohexylmethyl)-N-{2-[(2S)-1-methylpyrrolidin-2-yl]ethyl}-1,2,3,4-tetrahydroisoquinoline-7-sulfonamide difumarate monohydrate.

11.-12. (canceled)

13. The method according to claim 1, wherein the cholinesterase inhibitor is donepezil or a pharmaceutically acceptable salt thereof.

14. The method according to claim 13, wherein the donepezil or a pharmaceutically acceptable salt thereof is donepezil hydrochloride.

15. The method according to claim 1, wherein the treatment comprises a reduction in the worsening of cognitive function of the patient.

16. The method according to claim 1, wherein the treatment comprises a reduction in the worsening of functional capability of the patient.

17. The method according to claim 1, wherein the treatment comprises a reduction in the worsening of attention of the patient.

18. The method according to claim 1, wherein the treatment comprises a maintenance or reduction in the symptoms of apathy of the patient.

19. The method according to claim 1, wherein the treatment comprises an increase in patient responder rate based on a combined criterion, measured with ADAS-Cog and ADCS-ADL and CGIC.

20. The method according to claim 1, wherein the treatment comprises a maintenance in a sleep parameter selected from the group consisting of total sleep time, number of awakenings, interdaily variability and intradaily stability.

21. The method according to claim 1, wherein the treatment comprises an alteration in a sleep parameter selected from the group consisting of sleep fragmentation, sleep efficiency, wake after sleep onset, mean awakening duration and relative amplitude.

22. The method according to claim 1, wherein the treatment comprises an improvement in episodic memory of the patient.

23.-24. (canceled)

25. A method of treating Alzheimer's disease in a patient on stable therapy with a cholinesterase inhibitor and in need of said treatment, comprising administering to said patient a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof.

26. The method according to claim 25 wherein the cholinesterase inhibitor is donepezil or a pharmaceutically acceptable salt thereof.

27. A method for the symptomatic treatment of Alzheimer's disease comprising administering to a patient in need thereof a therapeutically effective amount of 2-(cyclohexylmethyl)-N-{2-[(2S)-1-methylpyrrolidin-2-yl]ethyl}-1,2,3,4-tetrahydroisoquinoline-7-sulfonamide or pharmaceutically acceptable salt thereof, wherein the treatment comprises treatment of one or more symptoms selected from the group consisting of disturbances of memory, disturbances of praxis, disturbances of attention, confusion, irritability and aggression, mood swings, language breakdown, long-term memory loss, withdrawal of the sufferer, loss of motor control, impairment of (cognitive) executive functions, functional disability, and apathy.

28. (canceled)
